(19) United States
(12) Patent Application Publication
(10) Pub. No.: US 2019/0209585 A1
(43) Pub. Date: Jul. 11, 2019

(54) COMBINATION THERAPY COMPRISING A POLYUNSATURATED KETONE AND A SECOSTEROID

(71) Applicant: Avexxin AS, Trondheim (NO)

(72) Inventors: Berit Johansen, Trondheim (NO); Astrid Jullumstrø Feuerherm, Trondheim (NO)

(21) Appl. No.: 16/306,106

(22) PCT Filed: Jun. 5, 2017
(86) PCT No.: PCT/EP2017/063625

§ 371 Date: Nov. 30, 2018

(30) Foreign Application Priority Data
Jun. 3, 2016 (GB) 1609721.4
Jul. 29, 2016 (GB) 1613172.4
Mar. 17, 2017 (GB) 1704279.7

(51) Int. Cl. A61K 31/573; A61K 45/06; A61P 17/06

(52) U.S. Cl. CPC A61K 31/573; A61K 31/5578; A61P 17/06; A61K 45/06

(57) ABSTRACT

A synergistic pharmaceutical composition for simultaneous, parallel, sequential or separate use comprising a polyunsaturated ketone, a secosteroid and, optionally, the corticosteroid partner betamethasone. The composition has utility in the treatment and prevention of skin disorders.

This invention relates to a pharmaceutical composition comprising certain polyunsaturated long-chain ketones in combination with certain secosteroids such as calcipotriol, tacalcitol or a pharmaceutically acceptable salt, or a hydrate or solvate thereof. The invention also relates to the use of said pharmaceutical composition for the treatment or prevention of skin conditions such as dermatitis and psoriasis.

### BACKGROUND

This invention is concerned with a combination therapy for the treatment of certain skin conditions such as psoriasis and dermatitis. In its broadest sense, dermatitis is inflammation of the skin. It is a common and disfiguring skin condition which requires quick and efficient treatment. Dermatitis symptoms vary, however, with the different forms of the condition. Symptoms vary from skin rashes to bumpy rashes through to flaky skin and blisters. Although different types of dermatitis have varying symptoms, there are certain signs that are common for all of them, including redness of the skin, swelling, itching, skin lesions and sometimes oozing and scarring.

Also, the area of the skin on which the symptoms appear tends to be different with every type of dermatitis. Types of dermatitis are classified according to the cause of the condition. Contact dermatitis is caused by an allergen or an irritating substance. Irritant contact dermatitis accounts for 80% of all cases of contact dermatitis.

Atopic dermatitis is very common worldwide and increasing in prevalence. Atopic dermatitis is a type of eczema and is an inflammatory, chronically relapsing, non-contagious and itchy skin disorder.

Other less common forms of dermatitis include dermatitis herpetiformis. It is characterized by intensely itchy, chronic papulovesicular eruptions, usually distributed symmetrically on extensor surfaces such as the back of neck, scalp, elbows, knees, back, hairline, groin or face.

Seborrheic dermatitis is a dermatitis that occurs in the vicinity of sebaceous glands and is caused by sebum over production. The condition tends to give a scaly, flaky skin condition.

Stasis dermatitis is an inflammation on the lower legs which is caused by build-up of blood and fluid and it is more likely to occur in people with varicose veins.

Other common skin disorders include psoriasis. This is an autoimmune induced, chronic disease of skin characterised by red, itchy and scaly skin patches. Skin disorders in general and dermatitis and psoriasis in particular are disfiguring and can lead to reluctance of a sufferer to let people see their condition. Successful treatments of these skin disorders are therefore sought.

A common treatment for skin disorders is administration of one or more topical secosteroids. The present inventors have now found that the combination of certain polyunsaturated ketones and certain secosteroids, such as calcipotriol and tacalcitol or a pharmaceutically acceptable salt, or a hydrate or solvate thereof results in a synergistic improvement in performance.

### SUMMARY OF INVENTION

Thus, viewed from one aspect the invention provides a pharmaceutical composition comprising:

(A) one or more compounds of formula (I):

R-L-CO—X (I)

wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;

L is a linking group forming a bridge of 1 to 5 atoms between the R group and the carbonyl CO wherein L comprises at least one heteroatom in the backbone of the linking group; and

X is an electron withdrawing group;

or a pharmaceutically acceptable salt, or a hydrate or solvate thereat and

(B) one or more secosteroid partners, preferably selected from the group consisting of calcipotriol, alfacalcidol, calcifediol, calcitriol, calcitroic acid, cholecalciferol, dihydrotachysterol, 24,25-dihydroxycholecalciferol, eldecalcitol, ergocalciferol, falecalcitriol, paricalcitol, previtamin D3, tacalcitol, 22-dihydroergocalciferol, sitocalciferol or a pharmaceutically acceptable salt, or a hydrate or solvate thereof, especially calcipotriol or tacalcitol or a pharmaceutically acceptable salt, or a hydrate or solvate thereof.

In a preferred embodiment, calcipotriol or a pharmaceutically acceptable salt, or a hydrate or solvate thereof is the secosteroid partner.

Viewed from another aspect the invention provides a pharmaceutical kit composition for simultaneous, in parallel, sequential or separate use comprising a first composition comprising at least one compound (I) as herein defined and a pharmaceutically-acceptable diluent or carrier, and a second composition comprising at least one compound (B) as the secosteroid partner herein defined such as calcipotriol or tacalcitol or a pharmaceutically acceptable salt, or a hydrate or solvate thereof and a pharmaceutically-acceptable diluent or carrier.

In particular, the invention relates to a pharmaceutical composition or kit as herein before defined in which the compound of formula (I) is:

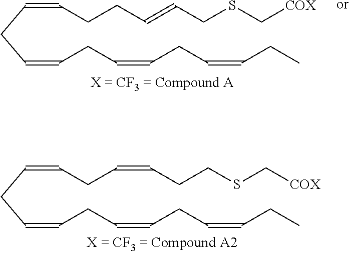

or a pharmaceutically acceptable salt, or a hydrate or solvate thereof. In particular, the secosteroid partner (B) is calcipotriol or tacalcitol or a salt, hydrate or solvate thereof.

At least one other secosteroid partner may be combined with the calcipotriol to achieve intended results, for example, 1 or 2 of such compounds. Alternatively, the calcipotriol (including a pharmaceutically acceptable salt, or a hydrate or solvate thereof) may be substituted by at least one other secosteroid partner, for example, 1 or 2 of such other compounds (including salts, hydrates and solvates of such compounds).

Viewed from another aspect the invention provides a pharmaceutical composition as hereinbefore defined for use in the treatment or prevention of a skin disorder such as psoriasis or dermatitis.

Viewed from another aspect the invention provides a method of treating or preventing a skin disorder such as psoriasis or dermatitis in a patient in an animal subject, for example, a mammal such as rodent (mouse, rat, rabbit), monkey (or other non-human primate), pig or other laboratory animal used as a model to study skin disorders. Another suitable mammalian subject is a need thereof. In one embodiment, the invention comprises administering to said subject (e.g. a human patient), an effective amount of a pharmaceutical composition as herein before defined.

Viewed from another aspect the invention provides a method of treating, such as reducing symptoms of, or preventing a skin disorder such as psoriasis or dermatitis, in a patient in need thereof comprising administering to said patient, preferably a human, an effective amount of at least one compound of formula (I) and simultaneously, in parallel, separately or sequentially administering to said patient an effective amount of at least one compound (B) (e.g., 1, 2 or 3 of such compounds) as herein defined. In sequential administration either compound can be administered first.

Viewed from another aspect the invention provides a method of treating, such as reducing symptoms of, or preventing a skin disorder such as psoriasis or dermatitis, in a patient in need thereof comprising:
(i) identifying a patient who has received either a compound of formula (I) or a compound (B);
   administering to said patient an effective amount of either at least one compound (B) as herein defined or at least one compound of formula (I) as herein before defined so that said patient is administered with both at least one compound of formula (I) and at least one compound (B).

In preferred embodiments, 1, 2 or 3 of compound B will be suitable for use with the invention with 1 or 2 of compound B being preferred for many invention applications.

Viewed from another aspect the invention provides use of a pharmaceutical composition as hereinbefore defined in the manufacture of a medicament for treating or preventing a skin disorder such as psoriasis or dermatitis.

Viewed from another aspect the invention provides a process for the preparation of a pharmaceutical composition as hereinbefore defined comprising blending at least one compound of formula (I) or a pharmaceutically acceptable salt, or a hydrate or solvate thereof and at least one compound (B) or a salt, hydrate or solvate thereof in the presence of at least one pharmaceutical excipient.

### Definitions

The term lower alkyl is used herein to refer to C1-6 alkyl groups, preferably C1-4 alkyl groups, especially C1-3 alkyl groups. These alkyl groups can be linear or branched, preferably linear.

In one embodiment, the invention relates to a pharmaceutical composition in which at least one compound (I) and at least one secosteroid partner (e.g., 1, 2, or 3 of such compounds) are blended together in a single composition. The invention also relates to a pharmaceutical composition in the form of a kit in which the active compounds are provided in separate compositions but are designed for administration simultaneously in parallel, separately or sequentially. Any method for treating or preventing a skin disorder as defined herein encompasses simultaneous, in parallel, separate or sequential administration of the active components or administration of the composition of the invention.

The pharmaceutical composition of the invention is a “combination”, which means either a fixed combination in one dosage unit form, or non fixed combination such as a kit of parts for combined administration where at least one compound of the formula (I) and at least one secosteroid partner(s) (e.g., 1, 2 or 3 of such compounds) may be administered independently at the same time (e.g. in parallel) or separately within time intervals, especially where these time intervals allow that the combination partners show a cooperative and preferably a synergistic effect.

Thus a “pharmaceutical composition” as used herein means a product suitable for pharmaceutical use that results from the mixing, admixing or combining more than one active ingredient and includes both fixed and non-fixed combinations of the active ingredients. The term “fixed combination” or “fixed dose” means that the active ingredients, e.g. a compound of formula (I) and a secosteroid partner such as calcipotriol, are both administered to a patient simultaneously in the form of a single entity or dosage. The pharmaceutical composition can also be a “non-fixed combination” which means that the active ingredients, e.g. a compound of formula (I) and the secosteroid partner are both administered to a patient as separate entities either simultaneously, in parallel, concurrently or sequentially with no specific time limits, wherein such administration provides therapeutically effective levels of the two compounds in the body of the animal in need thereof.

A secosteroid partner as used herein means a synthetic or semi-synthetic secosteroid generally suitable for intended goals of the invention. Preferred secosteroid partners include the following: calcipotriol, alfacalcidol, calcifediol, calcitriol, calcitroic acid, cholecalciferol, dihydrotachysterol, 24,25-dihydroxycholecalciferol, eldecalcitol, ergocalciferol, falecalcitriol, paricalcitol, previtamin D3, tacalcitol, 22-dihydroergocalciferol, sitocalciferol or a pharmaceutically acceptable salt, or a hydrate or solvate thereof. Calcipotriol or tacalcitol or a pharmaceutically acceptable salt, or a hydrate or solvate thereof are especially preferred secosteroid partners.

All discussion below relating to preferred compounds of the invention is equally applicable to both these aspects of the invention.

### DETAILED DESCRIPTION

This invention concerns a combination therapy of at least one compound of formula (I) and at least one secosteroid partner, in particular 1, 2 or 3 of such compounds with 1 or 2 compounds being preferred for many invention applications. In a preferred embodiment calcipotriol or tacalcitol or a salt, hydrate or solvate thereof is the secosteroid partner. We have surprisingly found that this combination therapy results in synergy. Our results demonstrate a reduction in the proliferation and viability of HaCaT cells, the pharmaceutical composition offering a larger decrease than could have been expected from the use of compounds individually, i.e. the combination of the compounds produces an overall effect that is greater than the sum of the individual elements.

### Pharmaceutical Composition of the Invention

The invention relies on the therapeutic combination of at least one compound of formula (I) or a pharmaceutically acceptable salt, or a hydrate or solvate thereof and at least one secosteroid partner such as calcipotriol or a pharmaceutically acceptable salt, or a hydrate or solvate thereof. The compound of formula (I) is

R-L-CO—X (I)

wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;

L is a linking group forming a bridge of 1 to 5 atoms between the R group and the carbonyl CO wherein L comprises at least one heteroatom in the backbone of the linking group; and

X is an electron withdrawing group; or a pharmaceutically acceptable salt, or a hydrate or solvate thereof.

The group R preferably comprises 5 to 9 double bonds, preferably 5 or 8 double bonds, e.g. 5 to 7 double bonds such as 5 or 6 double bonds. These bonds should be non-conjugated. It is also preferred if the double bonds do not conjugate with the carbonyl functionality.

The double bonds present in the group R may be in the cis or trans configuration however, it is preferred if the majority of the double bonds present (i.e. at least 50%) are in the cis configuration. In further advantageous embodiments all the double bonds in the group R are in the cis configuration or all double bonds are in the cis configuration except the double bond nearest the carbonyl group which may be in the trans configuration.

The group R may have between 10 and 24 carbon atoms, preferably 12 to 20 carbon atoms, especially 17 to 19 carbon atoms.

Whilst the R group can be interrupted by at least one heteroatom or group of heteroatoms, this is not preferred and the R group backbone preferably contains only carbon atoms.

The R group may carry up to three substituents, e.g. selected from halo, C₁₋₆ alkyl e.g. methyl, or C₁₋₆ alkoxy. If present, the substituents are preferably non-polar, and small, e.g. a methyl group. It is preferred however, if the R group remains unsubstituted.

The R group is preferably an alkylene group.

The R group is preferably linear. It preferably derives from a natural source such as a long chain fatty acid or ester. In particular, the R group may derive from AA, EPA or DHA.

Thus, viewed from another aspect the invention employs a compound of formula (I′)

R-L-CO—X (I′)

wherein R is a C₁₀₋₂₄ unsubstituted unsaturated alkylene group said group comprising at least 4 non-conjugated double bonds;

L is a linking group forming a bridge of 1 to 5 atoms between the R group and the carbonyl CO wherein L comprises at least one heteroatom in the backbone of the linking group; and

X is an electron withdrawing group or a salt thereof.

Ideally R is linear. R is therefore preferably an unsaturated C₁₀₋₂₄ polyalkylene chain.

The linking group L provides a bridging group of 1 to 5 backbone atoms, preferably 2 to 4 backbone atoms between the R group and the carbonyl, such as 2 atoms. The atoms in the backbone of the linker may be carbon and/or be heteroatoms such as N, O, S, SO, SO₂. The atoms should not form part of a ring and the backbone atoms of the linking group can be substituted with side chains, e.g. with groups such as C₁₋₆ alkyl, oxo, alkoxy, or halo.

Preferred components of the linking group are —CH₂—, —CH(C₁₋₆alkyl)-, —N(C₁₋₆alkyl)-, —NH—, —S—, —O—, —CH═CH—, —CO—, —SO—, —SO₂— which can be combined with each other in any (chemically meaningful) order to form the linking group. Thus, by using two methylene groups and an —S— group the linker —SCH₂CH₂— is formed. It will be appreciated that at least one component of the linker provides a heteroatom in the backbone.

The linking group L contains at least one heteroatom in the backbone. It is also preferred if the first backbone atom of the linking group attached to the R group is a heteroatom or group of heteroatoms.

It is highly preferred if the linking group L contains at least one —CH₂— link in the backbone. Ideally the atoms of the linking group adjacent the carbonyl are —CH₂—.

It is preferred that the group R or the group L (depending on the size of the L group) provides a heteroatom or group of heteroatoms positioned α, β, γ, or δ to the carbonyl, preferably β or γ to the carbonyl. Preferably the heteroatom is O, N or S or a sulphur derivative such as SO.

Highly preferred linking groups L therefore are —NH₂CH₂, —NH(Me)CH₂—, —SCH₂—, —SOCH₂—, or —COCH₂—

The linking group should not comprise a ring.

Highly preferred linking groups L are SCH₂, NHCH₂, and N(Me)CH₂.

Viewed from another aspect the invention employs a compound of formula (II)

R-L-CO—X (II)

wherein R is a linear C₁₀₋₂₄ unsubstituted unsaturated alkylene group said group comprising at least 4 non-conjugated double bonds;

L is —SCH₂—, —SOCH₂, or —SO₂CH₂—; and

X is an electron withdrawing group or a salt thereof.

The group X is an electron withdrawing group. Suitable groups in this regard include O—C₁₋₆ alkyl, CN, OCO₂—C₁₋₆ alkyl, phenyl, CHal₃, CHal₂H, CHalH₂ wherein Hal represents a halogen, e. g. fluorine, chlorine, bromine or iodine, preferably fluorine.

In a preferred embodiment the electron withdrawing group is CHal₃, especially CF₃.

Thus, preferred compounds of formula (I) are those of formula (III)

R-Y1-Y2-CO—X (III)

wherein R and X are as hereinbefore defined;

Y1 is selected from O, S, NH, N(C₁₋₆-alkyl), SO or SO₂ and

Y2 is (CH₂)ₙ or CH(C₁₋₆alkyl); or

where n is 1 to 3, preferably 1.

More, preferred compounds of formula (I) are those of formula (IV)

R-Y1-CH₂—CO—X (IV)

wherein R is a linear C₁₀₋₂₄ unsubstituted unsaturated alkylene group said group comprising at least 4 non-conjugated double bonds;

X is as hereinbefore defined (e.g. CF₃); and

Y1 is selected from O, S, SO or SO₂.

Highly preferred compounds for use in the invention are depicted below.

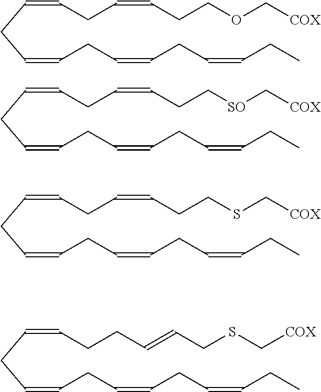

where X is as hereinbefore defined such as CF₃.

The following compounds are highly preferred for use in the invention:

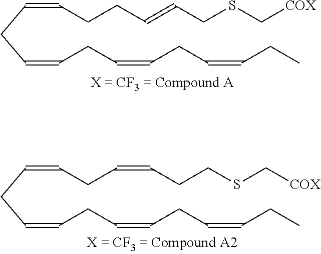

Salts, hydrates or solvates of any of these compounds could also be used. It will be appreciated that the pharmaceutical composition of the invention may comprise one or more than one compound of formula (I) as herein before defined, for example, 1, 2 or 3 of such compounds with 1 or 2 compounds being preferred for many invention applications.

### Secosteroid

The second component (compound B, i.e. the secosteroid partner) of the composition of the invention is a secosteroid, preferably a synthetic or semi-synthetic secosteroid, such as a non naturally occurring secosteroid, especially calcipotriol or tacalcitol or a pharmaceutically acceptable salt, or a hydrate or solvate thereof. Calcipotriol is a compound of formula:

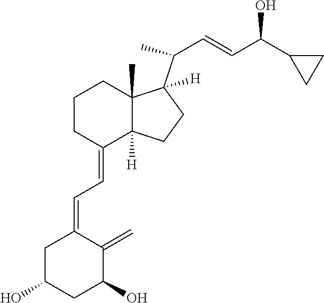 [SMILES: C=C1/C(=C\C=C2/CCC[C@]3(C)[C@@H]([C@H](C)/C=C/[C@@H](O)C4CC4)CC[C@@H]23)C[C@@H](O)C[C@@H]1O]

Tacalcitol is a compound of formula:

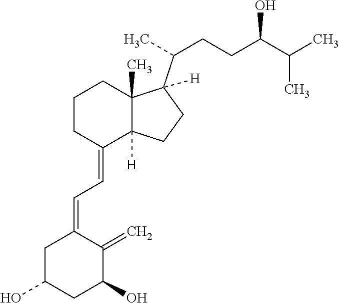 [SMILES: C=C1/C(=C\C=C2/CCC[C@]3(C)[C@@H]([C@H](C)CC[C@@H](O)C(C)C)CC[C@@H]23)C[C@@H](O)C[C@@H]1O]

In any composition of the invention the secosteroid may be present in a salt or non salt form. In particular, in any composition of the invention calcipotriol or tacalcitol may be present in a salt or non-salt form. If a salt form is used, any conventional salt form is possible. The salt may be a monosalt form, disalt or trisalt form, given the presence of multiple hydroxy groups on which salts can be formed.

Calcipotriol is a known commercial product and any known commercial form of calcipotriol can be used, such as calcipotriol hydrate. The calcipotriol is preferably in the form of its anhydrate or its monohydrate.

Tacalcitol is a known commercial product and any known commercial form of calcipotriol can be used tacalcitol monohydrate.

Whilst the invention is primarily described with reference to calcipotriol and tacalcitol, it is envisaged that other secosteroids could also be combined with the compounds of formula (I) to form synergistic combinations.

Vitamin D compounds are secosteroids and thus it is envisaged that component (B) may be selected from the group consisting of vitamins D₁, D₂, D₃, D₄ and D₅, or derivatives or analogues thereof. In particular, synthetic analogues of vitamin D are preferred, such as calcipotriol.

Possible further secosteroids include alfacalcidol, calcifediol, calcitriol, calcitroic acid, cholecalciferol, dihydrotachysterol, 24,25-dihydroxycholecalciferol, eldecalcitol, ergocalciferol, falecalcitriol, paricalcitol, previtamin D3, 22-dihydroergocalciferol, sitocalciferol, or pharmaceutically acceptable salts, or hydrates, or solvates thereof.

Preferred options include calcipotriol, calcitriol, falecalcitriol and tacalcitol, in particular calcipotriol and tacalcitol. Specific secosteroid compounds include calcipotriol hydrate and tacalcitol monohydrate, although any pharmaceutically acceptable salt, or hydrate or solvate thereof could be used.

The use of calcipotriol is especially preferred.

In one embodiment, the invention provides a pharmaceutical composition comprising:

(A) a compound of formula (I):

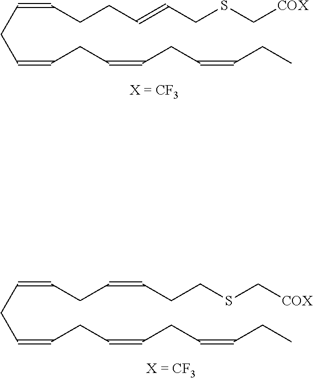

or a salt thereof; and

(B) a secosteroid partner selected from the group consisting of calcipotriol, alfacalcidol, calcifediol, calcitriol, calcitroic acid, cholecalciferol, dihydrotachysterol, 24,25-dihydroxycholecalciferol, eldecalcitol, ergocalciferol, falecalcitriol, paricalcitol, previtamin D3, tacalcitol, 22-dihydroergocalciferol, sitocalciferol or a pharmaceutically acceptable salt, or a hydrate or solvate thereof, especially tacalcitol or calcipotriol or a pharmaceutically acceptable salt, or a hydrate or solvate thereof, most especially calcipotriol or a pharmaceutically acceptable salt, or a hydrate or solvate thereof.

Alternatively, and as discussed above, the compositions of the invention could comprise calcipotriol or tacalcitol and additionally comprise one or more further secosteroids (e.g. 1, 2 or 3) to augment the properties of the composition of the invention. Suitable additional secosteroids include alfacalcidol, calcifediol, calcitriol, calcitroic acid, cholecalciferol, dihydrotachysterol, 24,25-dihydroxycholecalciferol, eldecalcitol, ergocalciferol, falecalcitriol, paricalcitol, previtamin D3, tacalcitol/calcipotriol, 22-dihydroergocalciferol, sitocalciferol or a pharmaceutically acceptable salt, or a hydrate or solvate thereof. Especially preferred is the combination of calcipotriol and tacalcitol or a pharmaceutically acceptable salt, or a hydrate or solvate thereof. Alternatively, one or more of the aforementioned secosteroids could be substituted for the tacalcitol/calcipotriol (including its salts and solvates thereof) so long as intended invention results are achieved.

It is also within the scope of the invention to combine the composition of the invention with other compounds conventionally used in conjunction with secosteroids such as calcipotriol in pharmaceuticals. The combination of calcipotriol with betamethasone is also a known therapy for psoriasis and hence the inclusion of betamethasone in the compositions of the invention is envisaged.

Viewed from another aspect therefore, the invention provides a pharmaceutical composition or kit as previously described further comprising betamethasone or a pharmaceutically acceptable salt, or a hydrate or solvate thereof.

In particular, we have found that the combination of a compound of formula (I), a secosteroid partner and betamethasone, in particular the combination of betamethasone, calciptriol and a compound of formula (I), e.g. compound A defined herein, leads to an unexpected synergistic effect. We demonstrate in the examples, that this combination leads to a synergistic reduction in cell viabilty making compounds of formula (I) ideal adjuvants to other drugs in treatment of the inflammation and itching caused by a number of skin conditions such as psoriasis.

It is also envisaged that the compound of formula (I) as defined herein and secosteroid partner such as calciptriol, could be combined with one or more corticosteriod partners in general, preferably selected from the group consisting of betamethasone, and dexamethasone or a pharmaceutically acceptable salt, or a hydrate or solvate thereof.

The amounts of each compound present in the composition of the invention are determined in molar terms, and the ratio of each is preferably secosteroid to compound of formula (I) of 10:1 to 1:10 moles, such as 5:1 to 1:5 moles, or such as 3:1 to 1:1 moles.

The amount of the compounds of the invention in the composition will often be determined by the physician depending on the dosage required.

### Skin Disorders

As noted above, the invention targets skin disorders, especially psoriasis and dermatitis. In particular, it is envisaged that the compositions of the invention may reduce inflammation and/or itchiness associated with the skin condition in question.

Viewed from another aspect the invention provides a method of treating, such as reducing symptoms of, or preventing inflammation of the skin in an animal subject in need thereof comprising administering to said animal an effective amount of a composition as herein defined.

In particular, the treatment of inflammation can occur via the reduction in expression of key inflammation markers of one or more of prostaglandin E2 (PGE2), interleukin-1beta (IL-1β), tumor necrosis factor (TNF) or interleukin-6 (IL-6). This may lead therefore to utility in animal subjects having or suspected of having psoriasis or dermatitis. The inventors have demonstrated that the composition of the invention, in particular the combination of compound A and calciptroil offers syngeristic reduction in the expression of PGE2, IL-1beta, TNF and IL-6.

The combination therapy of the invention may have utility in treating a variety of different forms of dermatitis, such as atopic dermatitis or contact dermatitis. Thus, the compounds of the invention may be used to treat contact dermatitis such as allergic contact dermatitis or irritant contact dermatitis.

The nature of the allergan or irritant which causes the contact dermatitis can vary a lot and many people have different reactions to different allergans/irritants.

One of the most common causes of allergic contact dermatitis are plants of the Toxicodendron genus: poison ivy, poison oak, and poison sumac. Certain alkyl resorcinols such as bilobol found in Gingko biloba fruits are strong skin irritants. Other allergens include nickel, gold, balsam of Peru (Myroxylon pereirae), and chromium.

Common causes of irritant contact dermatitis are harsh (highly alkaline) soaps, detergents, and cleaning products. Irritant contact dermatitis can be divided into forms caused by chemical irritants and those caused by physical irritants. Common chemical irritants implicated include solvents (alcohol, xylene, turpentine, esters, acetone, ketones, and others); metalworking fluids (neat oils, water-based metalworking fluids with surfactants); latex; kerosene; ethylene oxide; surfactants in topical medications and cosmetics (sodium lauryl sulfate); alkalies (drain cleaners, strong soap with lye residues). Physical irritant contact dermatitis may most commonly be caused by low humidity from air conditioning. Also, many plants directly irritate the skin.

A further form of contact dermatitis is photocontact dermatitis. The skin condition is caused by exposure to ultraviolet light (320-400 nm UVA).

The invention may also lead to a treatment of atopic dermatitis. Atopic dermatitis is a type of eczema and is an inflammatory, chronically relapsing, non-contagious and itchy skin disorder.

Other less common forms of dermatitis to be treated include dermatitis herpetiformis, seborrheic dermatitis and stasis dermatitis.

By treating or treatment is meant at least one of:

(i). inhibiting the disease i.e. arresting, reducing or delaying the development of the disease or a relapse thereof or at least one clinical or subclinical symptom thereof, or

(ii). relieving or attenuating one or more of the clinical or subclinical symptoms of the disease.

By prevention is meant (i) preventing or delaying the appearance of clinical symptoms of the disease developing in a mammal.

The benefit to a subject to be treated is either statistically significant or at least perceptible to the patient or to the physician. In general a skilled man can appreciate when “treatment” occurs. It is particularly preferred if the pharmaceutical compositions of the invention are used therapeutically, i.e. to treat a condition which has manifested rather than prophylactically. It may be that the pharmaceutical composition of the invention is more effective when used therapeutically than prophylactically.

The pharmaceutical composition of the invention can be used on any animal subject, in particular a mammal and more particularly a human or an animal serving as a model for a disease (e.g., rat, mouse, pig, monkey, etc.). For example, in one use a pharmaceutical composition of the invention is used as a positive control in the animal subject to test other compounds for activity and/or side effects.

In order to treat a disease an effective amount of the active pharmaceutical composition needs to be administered to a patient. A “therapeutically effective amount” means the amount of a pharmaceutical composition that, when administered to an animal for treating a state, disorder or condition, is sufficient to effect such treatment. The “therapeutically effective amount” will vary depending on the pharmaceutical composition, the disease and its severity and the age, weight, physical condition and responsiveness of the subject to be treated and will be ultimately at the discretion of the attendant doctor.

It may be that to treat skin disorders according to the invention that the pharmaceutical composition of the invention has to be readministered at certain intervals. Suitable dosage regimes can be prescribed by a physician.

The pharmaceutical composition of the invention typically comprises the active components in admixture with at least one pharmaceutically acceptable carrier selected with regard to the intended route of administration and standard pharmaceutical practice.

The term “carrier” refers to a diluent, excipient, and/or vehicle with which an active compound is administered. The pharmaceutical compositions of the invention may contain combinations of more than one carrier. Such pharmaceutical carriers are well known in the art. The pharmaceutical compositions may also comprise any suitable binder(s), lubricant(s), suspending agent(s), coating agent(s), and/or solubilizing agent(s) and so on. The pharmaceutical composition can also contain other active components, e.g. other drugs for the treatment of skin disorders.

It will be appreciated that pharmaceutical compositions for use in accordance with the present invention may be in the form of oral, parenteral, transdermal, sublingual, topical, implant, nasal, or enterally administered (or other mucosally administered) suspensions, capsules or tablets, which may be formulated in conventional manner using one or more pharmaceutically acceptable carriers or excipients. The pharmaceutical compositions of the invention could also be formulated as nanoparticle formulations.

However, for the treatment of skin disorders, the pharmaceutical composition of the invention will preferably be administered topically. The pharmaceutical composition may therefore be provided in the form of a cream, gel, foam, salve or ointment.

The pharmaceutical composition of the invention may contain from 0.01 to 99% weight—per volume of the active material. The therapeutic doses will generally be between about 10 and 2000 mg/day and preferably between about 30 and 1500 mg/day of active components combined. Other ranges may be used, including, for example, 50-500 mg/day, 50-300 mg/day, 100-200 mg/day or active components combined.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

The invention is described further below with reference to the following non-limiting examples and figures.

### DESCRIPTION OF FIGURES

FIG. 1 shows the results of the combination therapy of the invention. Co-treatment with cPLA2α inhibitor Compound A and Calcipotriol hydrate shows synergistic effects on decreasing keratinocyte cell proliferation and viability compared to each inhibitor alone. Average and standard deviation of 2-4 independent experiments performed in series of 8 technical replicates per treatment.

FIG. 2 shows co-treatment with corticosteroid betamethasone and vitamin D analogue calcipotriol shows synergistic effects on keratinocyte cell proliferation and viability compared to each inhibitor alone. Average and standard deviation of 2-4 independent experiments performed in series of 8 technical replicates per treatment. The use of betamethasone and calcipotriol is a known synergistic psoriasis treatment. FIG. 2 is added to show that the results of the present invention are comparable to the results in FIG. 2, proving the presence of synergy.

FIG. 3 shows a dose response of Compound A on immortalized keratinocyte cell line HaCat cell viability. Data presented are average and standard deviation of 3 independent experiments performed in series of 8 technical replicates per treatment. Star (*) represent significant difference compare to control (100%) (*P≤0.05; **P≤0.01; ***P≤0.001; ****P≤0.0001).

FIG. 4 shows dose response of calcipotriol on immortalized keratinocyte cell line HaCat cell viability. Data presented are average and standard deviation of 3 independent experiments performed in series of 8 technical replicates per treatment. Star (*) represent significant difference compare to control (100%) (*P≤0.05; **P≤0.01; ***P≤0.001; ****P≤0.0001).

FIG. 5 shows that co-treatment with compound A and calcipotriol has synergistic effects on human keratinocyte cell viability compared to each inhibitor alone. Data presented are average and standard deviation of 3 independent experiments performed in series of 8 technical replicates per treatment. Star (*) represent significant difference in compare to control (100%) and in between inhibitors indicated with bars (*P≤0.05; **P≤0.01; ***P≤0.001; ****P≤0.0001).

FIG. 6 shows co-treatment of Compound A with Calcipotriol and Betamethasone has a synergistic effect on human keratinocyte cell viability. Data presented are average and standard deviation of 1 independent experiments performed in series of 8 technical replicates per treatment. Star (*) represent significant difference in compare to control (100%) and in between inhibitors indicated with bars (*P≤0.05; **P≤0.01; ***P≤0.001; ****P≤0.0001).

FIG. 7 shows that LPS induces PGE2 production in PBMC challenged with LPS. Compound A dose-dependently reduce PGE2 production. When suboptimal doses of Compound A are combined with a non-effective dose of calcipotriol, synergistic reduction of PGE2 levels are seen. Ctrl; untreated PBMC (no LPS, no inhibitor), Compound A, CAL: calcipotriol. Results shown for one blood donor experiment.

FIG. 8 shows that LPS induces cytokine production in PBMC challenged with LPS. Compound A dose-dependently reduce cytokine production, including IL-1β, TNF and IL-6.

### EXAMPLE 1

The following compounds were used in the Experiments:

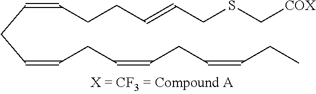

### Co-Treatment Compound A & Calcipotriol:

### Methods:

### Cell Culture:

The spontaneously immortalized, nontumorigenic skin keratinocyte cell line HaCaT was maintained in DMEM supplemented with 5% (v/v) FBS, 0.3 mg/ml glutamine and 0.1 mg/ml gentamicin at 37° C. with 5% CO₂ in a humidified atmosphere. Subculture using trypsin-EDTA was performed every 3-4 days with split ratio of 1:3-1:4 to ensure actively proliferating cells.

### Resazurin Assay:

Cells were seeded in 96 well plates in fully supplemented medium at a density of 2500 cells per well. Following 72 hours of cultivation, the cells were starved of serum in 0.25% FBS/DMEM overnight to halt proliferation, synchronize and to increase cell sensitivity to treatment. On day 4, the cells were treated with cPLA2α inhibitor Compound A and vitamin D analogue Calcipotriol hydrate (Sigma Aldrich #C4369) and left to incubate for 2 hour in incubator at 37° C. with 5% CO₂ in a humidified atmosphere before fluorescence was read at 544 nm excitation and 590 nm emission wavelength. The cells were observed under the microscope to evaluate possible morphology changes and signs of stress before addition of resazurin. The experiments were performed in series of 8 wells per treatment and repeated 2-3 times.

### Results:

Co-treatment with cPLA2α inhibitor Compound A and vitamin D analogue Calcipotriol shows synergistic effects on decreasing keratinocyte cell proliferation and viability compared to each inhibitor alone.

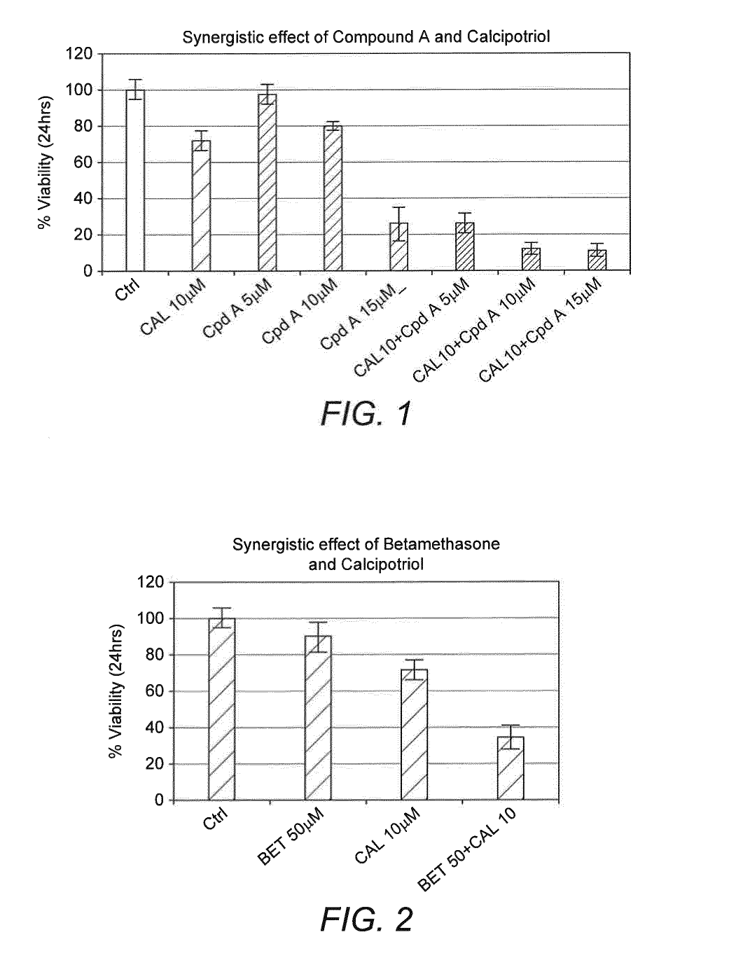

Initial experiments were performed to determine dose response of Compound A alone. The inhibitor reduced cell proliferation and viability slightly at 10 μM, whereas 5 μM did not show any affect (FIG. 1). On this basis, combination treatment experiments were designed in which sub-effective doses of the Compound A inhibitor and Calcipotriol were combined.

Following 24 hours of treatment, 10 μM of Calcipotriol and 5 or 10 μM Compound A alone showed little or no effect on reducing proliferation and viability of HaCaT cells, whereas 15 μM Compound A clearly reduced viability by ˜70%. However, when combining the sub-effective 5 and 10 μM doses of Compound A and Calcipotriol, a significant ˜70% and ˜90% reduction of proliferation and viability was observed (FIG. 1). This observed trend of synergistic effects on cell proliferation and viability indicates beneficial effects of co-treatment of on skin disorders.

Several key pathways are dysregulated in skin disorders such as psoriasis and atopic dermatitis. With this preliminary result, cPLA2α inhibitors represent a promising adjuvant treatment to other drugs in treatment of the inflammation and itching caused by a number of skin conditions such as psoriasis and dermatitis.

### EXAMPLE 2

### Co-Treat Betamethasone & Calcipotriol

### Methods:

### Cell Culture:

The spontaneously immortalized, nontumorigenic skin keratinocyte cell line HaCaT was maintained in DMEM supplemented with 5% (v/v) FBS, 0.3 mg/ml glutamine and 0.1 mg/ml gentamicin at 37° C. with 5% CO₂ in a humidified atmosphere. Subculture using trypsin-EDTA was performed every 3-4 days with split ratio of 1:3-1:4 to ensure actively proliferating cells.

### Resazurin Assay:

Cells were seeded in 96 well plates in fully supplemented medium at a density of 2500 cells per well. Following 72 hour of cultivation, the cells were starved of serum in 0.25% FBS/DMEM overnight to halt proliferation, synchronize the cells and to increase cell sensitivity to treatment. On day 4, the cells were treated with corticosteroid Betamethasone 17, 21-dipropionate (Sigma Aldrich #B1152) and vitamin D analogue Calcipotriol hydrate (Sigma Aldrich #C4369) for 24 hours. On day 5, resazurin was added according to the manufacturer's instruction (RnD Systems, UK) and left to incubate for 2 hour in incubator at 37° C. with 5% CO₂ in a humidified atmosphere before fluorescence was read at 544 nm excitation and 590 nm emission wavelength. The cells were observed under the microscope to evaluate possible morphology changes and signs of stress before addition of resazurin. The experiments were performed in series of 8 wells per treatment and repeated 2-3 times.

### Results:

Co-treatment with corticosteroid Betamethasone and vitamin D analogue Calcipotriol shows synergistic effects on decreasing keratinocyte cell proliferation and viability compared to each inhibitor alone.

Betamethasone and Calcipotriol combination has already been established in treatment of Psoriasis. We here tested this established co-treatment to verify our methodology. Following 24 hours of treatment, 50 μM of Betamethasone and 10 μM Calcipotriol alone showed a 10% and 20% reduction of cell proliferation and viability which increased to a ˜35% reduction when given in combination (FIG. 2). This observed trend of synergistic effects on cell proliferation and viability show the relevance of the rezasurin assay and validate the previously reported beneficial effects of Betamethasone and Calcipotriol co-treatment on skin disorders.

### EXAMPLE 3

Compound A and Calcipotriol show dose response on immortalized keratinocyte cell line HaCat cell viability.

### Cell Culture:

The spontaneously immortalized, nontumorigenic skin keratinocyte cell line HaCaT was maintained in DMEM supplemented with 5% (v/v) FBS, 0.3 mg/ml glutamine and 0.1 mg/ml gentamicin at 37° C. with 5% CO₂ in a humidified atmosphere. Subculture using trypsin-EDTA was performed every 3-4 days with split ratio of 1:3-1:4 to ensure actively proliferating cells.

### Resazurin Assay:

Cells were seeded in 96 well plates in fully supplemented medium at a density of 3000 cells per well. Following 48-72 hour of cultivation, the cells were starved of serum in 0.25% FBS/DMEM overnight to halt proliferation, synchronize the cells and to increase cell sensitivity to treatment. Next day, the cells were treated with compound A or calcipotriol for 24 hours. Resazurin was added next day according to the manufacturer's instruction (RnD Systems, UK) and left to incubate for 2 hour in incubator at 37° C. with 5% CO₂ in a humidified atmosphere before fluorescence was read at 544 nm excitation and 590 nm emission wavelength. The cells were observed under the microscope to evaluate possible morphology changes and signs of stress before addition of resazurin. The experiments were performed in series of 8 wells per treatment and repeated 2-3 times.

### Results

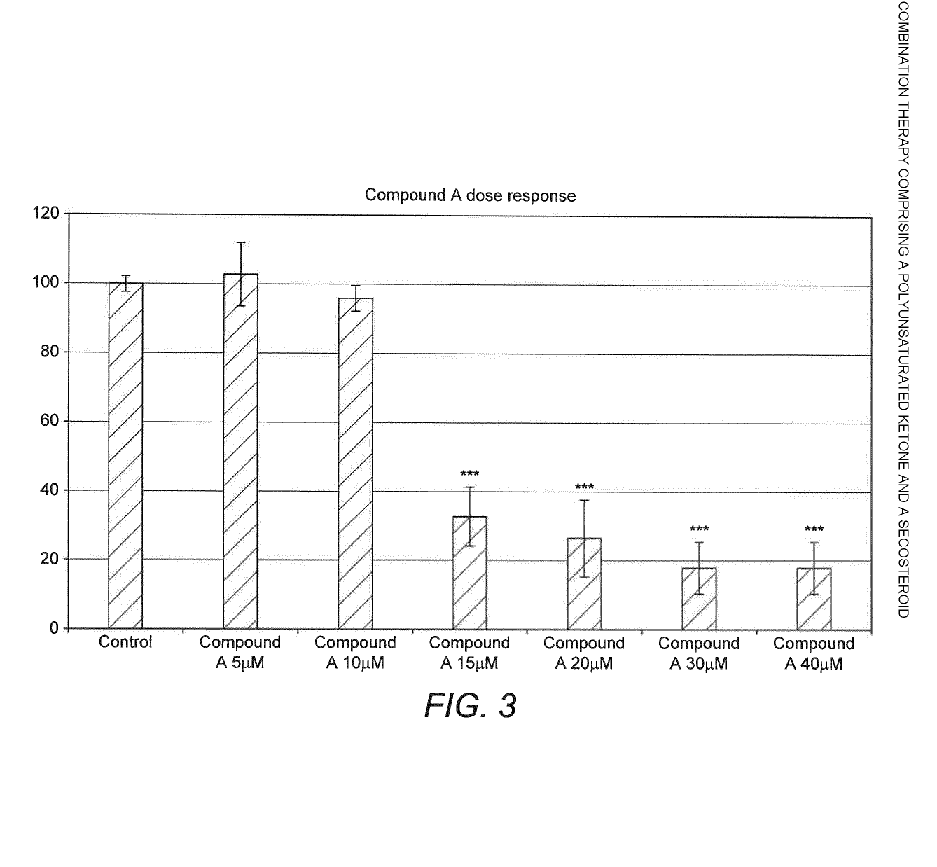

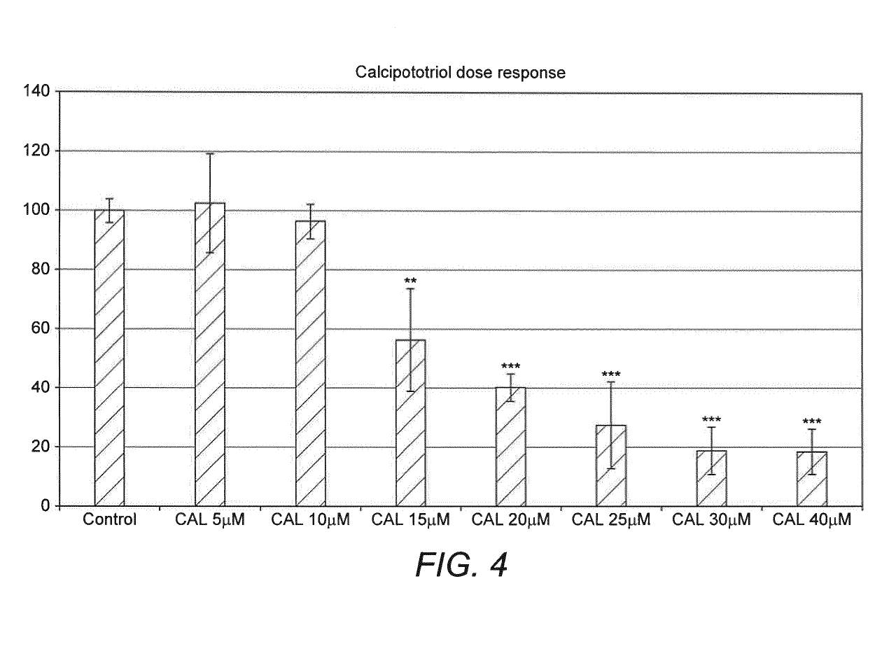

In this study, experiments were performed to determine dose response of Calcipotriol and compound A. Compound A and calcipotriol were found to affect cell viability at 15 μM, whereas at doses 1-10 μM no signs of impairment in cell viability were observed (FIG. 3/4).

### EXAMPLE 4

Co-treatment with compound A and calcipotriol shows synergistic effects on immortalized keratinocyte cell line HaCat cell viability compared to each inhibitor alone.

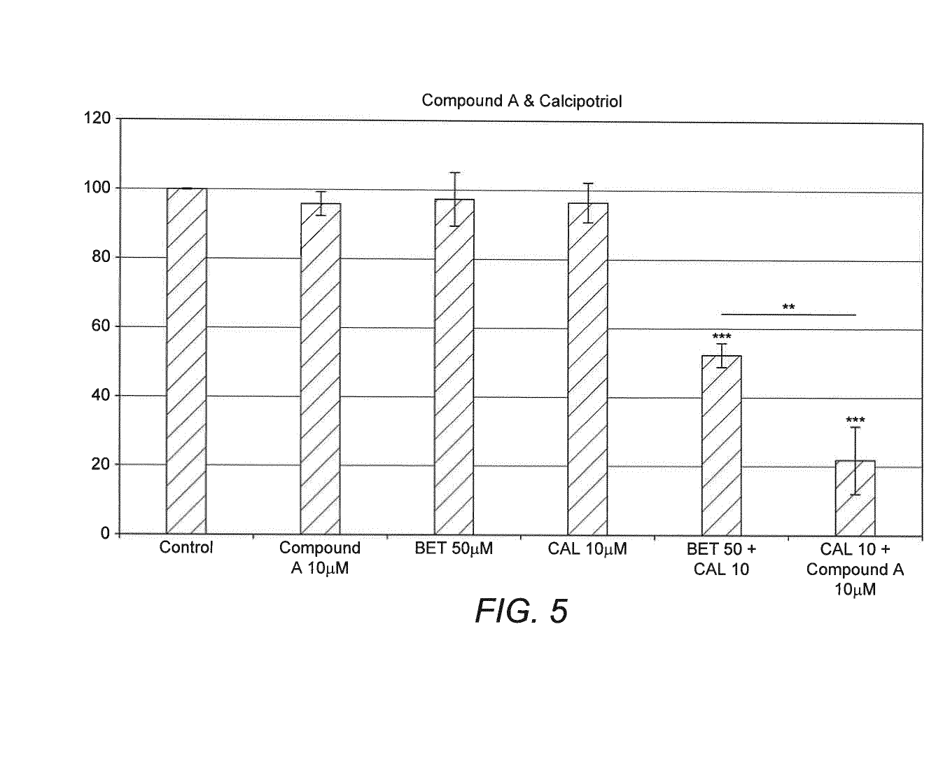

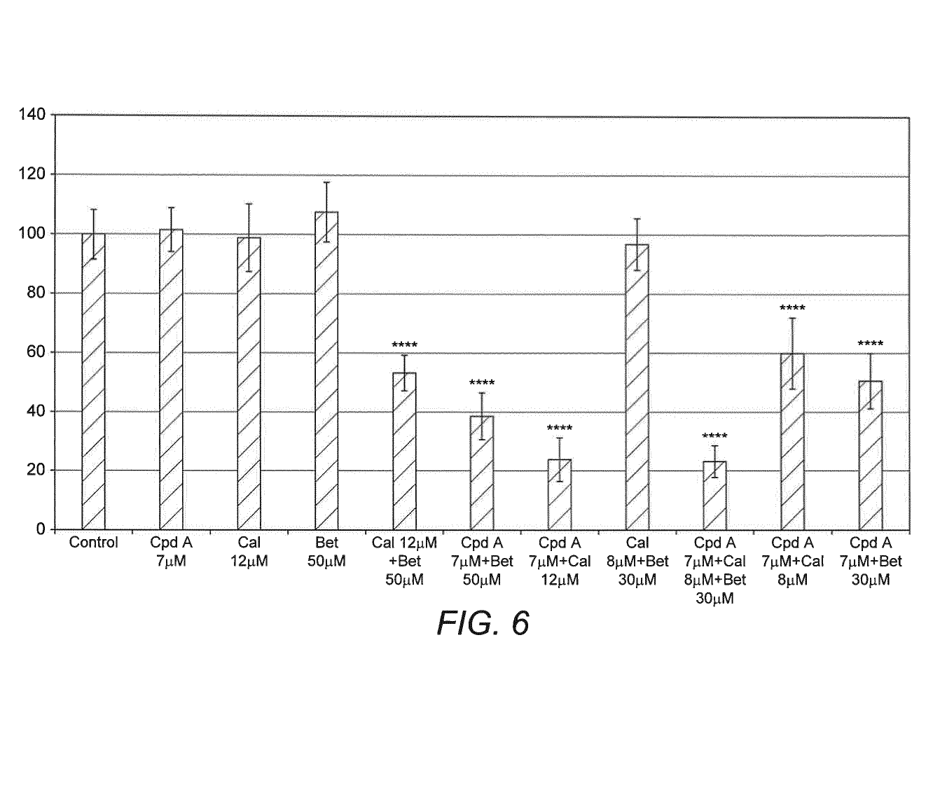

As noted in FIG. 3/4, initial experiments were performed to determine dose response of compound A and calcipotriol alone (FIG. 3/4). Both of them shows reduced cell viability to the cells at 15 μM, whereas at 10 μM no signs of impairment in cell viability was found (FIG. 3/4). On this basis, combination treatment was designed in which sub-effective doses of the inhibitor compound A (10 μM) and calcipotriol (10 μM) were combined. Combination of compound A and calcipotriol were also compared with already established combination of betamethasone and calcipotriol. Following 24 hours of treatment, 10 μM of calcipotriol and 50 μM of betamethasone shows 45% reduction of cell viability which increased to nearly 70% when same concentration of calcipotriol is given with compound A 10 μM (FIG. 5). This observed trend of synergistic effects on cell viability indicates better beneficial effects calcipotriol combination with compound A than betamethasone dipropionate on skin disorders.

### EXAMPLE 5

Co-Treatment of Compound A with Vitamin D Analogue Calcipotriol and Corticosteroid Hormone Receptor Agonist Betamethasone Shows Synergistic Effects on Immortalized Keratinocyte Cell Line HaCat Viability Both in Dual and Triple Combination in Compared to Each Inhibitor Alone

### Cell Culture:

The spontaneously immortalized, nontumorigenic skin keratinocyte cell line HaCaT was maintained in DMEM supplemented with 5% (v/v) FBS, 0.3 mg/ml glutamine and 0.1 mg/ml gentamicin at 37° C. with 5% CO₂ in a humidified atmosphere. Subculture using trypsin-EDTA was performed every 3-4 days with split ratio of 1:4 to ensure actively proliferating cells.

### Resazurin Assay:

Cells were seeded in 96 well plates in fully supplemented medium at a density of 3000 cells per well. Following 72 hour of cultivation, the cells were starved of serum in 0.25% FBS/DMEM overnight to halt proliferation, synchronize the cells and to increase cell sensitivity to treatment. Next day, the cells were treated with Compound A, vitamin D analogue Calcipotriol and corticosteroid hormone receptor agonist Betamethasone dipropionate for 24 hours. Resazurin was added next day according to the manufacturer's instruction (RnD Systems, UK) and left to incubate for 2 hour in incubator at 37° C. with 5% CO₂ in a humidified atmosphere before fluorescence was read at 544 nm excitation and 590 nm emission wavelength. The cells were observed under the microscope to evaluate possible morphology changes and signs of stress before addition of resazurin. The experiments were performed in series of 8 wells per treatment and repeated 2-3 times.

### Results:

Initial experiments were performed to determine dose response of Compound A and Calcipotriol and Betamethasone alone. Combination treatment was designed in which sub-optimal doses of the inhibitor Compound A and Calcipotriol and Betamethasone were combined. Combination of Compound A with Calcipotriol and Betamethasone were compared with already established combo of Betamethasone and Calcipotriol. Following 24 hours of treatment, 12 μM of Calcipotriol and 50 μM of Betamethasone shows 45% reduction of cell viability which increased to nearly 80% when same concentration of Calcipotriol is given with Compound A 7 μM. In addition, combination of Compound A 7 μM with 50 μM of Betamethasone cause 60% reduction.

In the same way, Calcipotriol 8 μM and Betamethasone 30 μM does not have any effect on cell viability. Nevertheless, addition of 7 μM to that dual combination cause almost 80% reduction, which is far better than the dual combination of same doses of Calcipotriol and Betamethasone with similar dose of Compound A.

These results show that Compound A can be used as adjuvant treatment to other drugs in treatment of the inflammation and itching caused by a number of skin conditions such as psoriasis.

### EXAMPLE 6

### Methods

Blood from healthy, consenting blood donors was collected at St. Olays Hospital HF, Trondheim, Norway.

### Isolation and Cell Culture of Peripheral Blood Mononuclear Cells.

From each donor, 2×6 ml heparin tubes with blood were used. Peripheral blood mononuclear cells (PBMC) were isolated using 50 mL SepMate™ tubes (STEMCELL™ Technology, UK) and Lymphoprep™ (Fresenius Kabi, N) density gradient according to the manufacturers instructions.

PBMC were resuspended in RPMI-1640 cell culture medium complemented with 5% heat-inactivated fetal bovine serum (FBS), 0.03% glutamine, 0.01 mg/mL gentamicin and 3.4 ppm β-mercaptoethanol (all reagents from Sigma-Aldrich). 1×10⁶ PBMC were seeded per well in 24-well plates and pre-treated for 2 hrs with Compound A and calcipotriol prior to the addition of Lipopolysaccaride (LPS, 10 ng/mL, total volume pr. well 1000 pt). Cells were incubated for 72 hrs (5% CO2, 37° C.) before the cells were centrifuged (10 min, 2000 rpm, 4° C.) to clear the supernant of cells. The supernatant were aliquoted and stored at −80° C. until analysis.

### PGE2 Analysis

PGE2 enzyme immunoassay analysis of PBMC supernatants was performed according to the kit protocol (Cayman Chemicals, #514010, USA). Samples were diluted 1:100 except for the untreated control PBMC supernatant that was assayed undiluted. The samples were hybridized overnight (18 h, 4° C.). The plate was read at OD 420 nm using a Cytation 5 imaging reader from BioTek and data acquired with the corresponding software Gen5 2.09. A four-parameter logistic model was fit to the absorbance data to determine PGE2 levels using the Cayman PGE2 protocol. PGE2 levels for all treatments are shown relative to the untreated PBMC supernatant.

### Results

As described previously, in vitro cellular experiments using HaCaT keratinocytes have shown significant synergistic effects on reducing keratinocyte viability when sub-optimal doses of Compound A and calcipotriol are combined. However, the viability assays performed to date cannot always reveal if the combination treatment may also yield synergistic anti-inflammatory effects, locally or systemically. Therefore, it was of interest to evaluate the effects of the Compound A and calciptriol combination in another physiologically and clinically relevant model system with an anti-inflammatory read-out.

Accordingly, we isolated peripheral blood mononuclear cells, treated them with various doses and combinations of Compound A and calcipotriol and finally added LPS as a trigger to mount a broad inflammatory response. The cell supernatants collected following 72 hrs of LPS stimulation were next analyzed with respect to levels of PGE2, a key proinflammatory mediator close to the cPLA2α target.

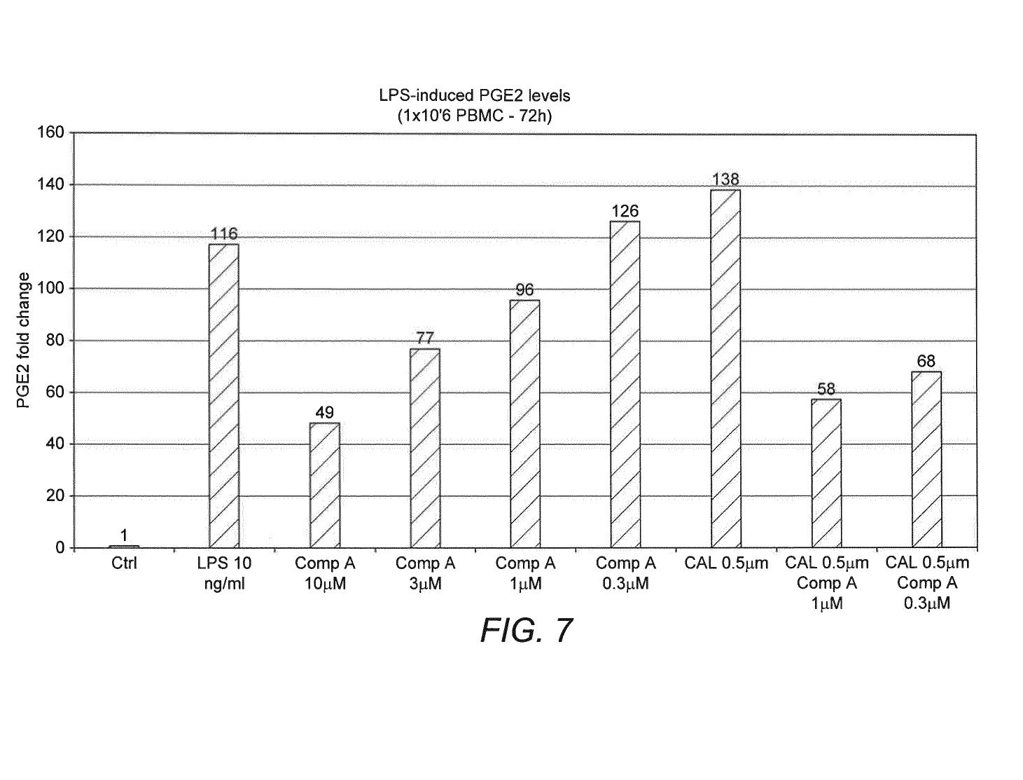

LPS clearly induced PGE2 production, confirming that cPLA2α and COX2 are activated by LPS (FIG. 7). PGE2 levels in untreated PBMC were in the 120-200 pg/mL range. LPS induced levels to varying degree in different donors to ˜10-47 000 pg/mL, i.e. ˜80-200 fold induction, which indicates considerable individual variation.

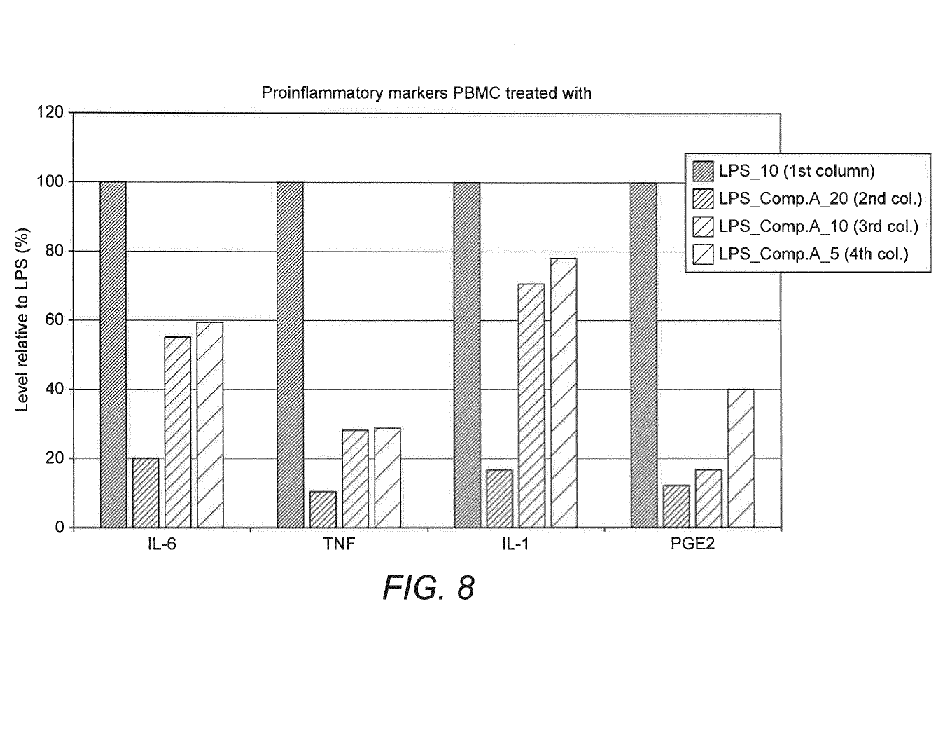

In addition to PGE2, LPS also induced a more general proinflammatory PBMC profile, including production of the cytokines TNF, IL-1β and IL6 (FIG. 8). PBMC cytokine production was dose-dependently reduced by Compound A, showing that the cPLA2c enzyme is regulating signaling leading to LPS-induced cytokine production.

In response to Compound A alone, a clear dose-dependent reduction in PGE2 levels were found, with an estimated IC50 of ˜10 μM. Compound A in doses 0.3 μM and 1 μM were found to be sub-optimal with little or no inhibition and were chosen for subsequent combo-experiments. In response to high doses of calcipotriol alone (<0.5 μM), PGE2 levels increased, indicating that calcipotriol may act as a stressor above a given threshold dose. The suboptimal calcipotriol dose 0.5 μM was chosen for inclusion in combo-experiments with Compound A.

When suboptimal doses of Compound A and calcipotriol were combined, the reduction of PGE2 was better than for each inhibitor alone; Compound A at 1 μM reduced PGE2 by 18%, Compound A at 0.3 μM showed no inhibition, and calcipotriol at 0.5 μM increased PGE2 by 18%. In contrast, the combination of Compound A (1 μM) with calcipotriol (0.5 μM) reduced PGE2 by 51%. The combination of Compound A (0.3 μM) with calcipotriol (0.5 μM) also reduced PGE2 levels by 42%, indicating synergistic effects of the combination compared to each inhibitor alone.

The synergy found with the Compound A and calcipotriol combination on PGE2 production in PBMC challenged with LPS, indicates that in addition to the synergistic effects on keratinocyte viability, beneficial systemic, clinically relevant, anti-inflammatory effects may be achieved as well.

The combination of Compound A and calcipotriol represents a novel, non-steroidal treatment for psoriasis and related diseases that offers improved efficacy at low doses and less adverse effects compared to existing therapeutic modalities.

1. A pharmaceutical composition comprising:
(A) at least one compound of formula (I):
R-L-CO—X (I)
wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;
L is a linking group forming a bridge of 1 to 5 atoms between the R group and the carbonyl CO wherein L comprises at least one heteroatom in the backbone of the linking group; and
X is an electron withdrawing group;
or a pharmaceutically acceptable salt, or a hydrate or solvate thereof; and
(B) one or more secosteroid partners, preferably selected from the group consisting of calcipotriol, alfacalcidol, calcifediol, calcitriol, calcitroic acid, cholecalciferol, dihydrotachysterol, 24,25-dihydroxycholecalciferol, eldecalcitol, ergocalciferol, falecalcitriol, paricalcitol, previtamin D3, tacalcitol, 22-dihydroergocalciferol, sitocalciferol or a pharmaceutically acceptable salt, or a hydrate or solvate thereof, especially calcipotriol or tacalcitol or a pharmaceutically acceptable salt, or a hydrate or solvate thereof.

2. A composition as claimed in claim 1 wherein the composition is a fixed combination or non-fixed combination.

3. A pharmaceutical composition as claimed in claim 1 for simultaneous, parallel, sequential or separate use comprising a kit comprising a first composition comprising at least one compound (I) and a pharmaceutically-acceptable diluent or carrier, and a second composition comprising at least one compound (B) and a pharmaceutically-acceptable diluent or carrier.

4. A composition as claimed in claim 1 wherein the compound (B) is calcipotriol, tacalcitol, calcitriol or falecalcitriol, preferably calcipotriol or tacalcitol or a pharmaceutically acceptable salt, or a hydrate or solvate thereof.

5. A composition as claimed in claim 1 wherein the compound (B) is calcipotriol or a pharmaceutically acceptable salt, or a hydrate or a solvate thereof.

6. A composition as claimed in claim 1 wherein the compound (B) is calcipotriol hydrate.

7. A composition as claimed in claim 1 wherein in formula (I), the group X is CHal₃, preferably CF₃.

8. A composition as claimed in claim 1 wherein in formula (I), the group R is a linear unsubstituted C₁₀₋₂₄ unsaturated alkylene group comprising at least 4 non-conjugated double bonds.

9. A composition as claimed in claim 1 wherein L is —SCH₂—.

10. A composition as claimed in claim 1 wherein said compound of formula (I) has the formula: 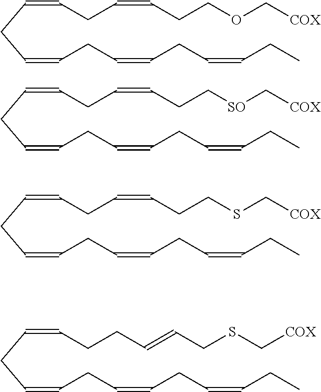
wherein X is as defined in claim 1, e.g. CF₃.

11. A composition as claimed in claim 1 wherein the compound of formula (I) is Compound A or Compound A2: 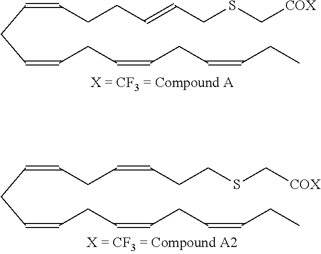
especially when compound (B) is calcipotriol or tacalcitol or a salt, hydrate or solvate thereof.

12. A composition as claimed in claim 1 wherein the molar ratio of compound (A) to (B) in the composition is 10:1 to 1:10, preferably 1:5 to 5:1.

13. A composition as claimed in claim 1 further comprising betamethasone or a pharmaceutically acceptable salt, or a hydrate or solvate thereof.

14. A composition as claimed in claim 13 comprising compound A, betamethasone or a pharmaceutically acceptable salt, or a hydrate or solvate thereof and calcipotriol or a pharmaceutically acceptable salt, or a hydrate or solvate thereof.

15. (canceled)

16. A method of treating, such as reducing symptoms of, or preventing a skin disorder such as psoriasis or dermatitis in a patient in need thereof comprising administering to said patient, preferably a human, an effective amount of a composition as claimed in claim 1.

17. A method of treating, such as reducing symptoms of, or preventing a skin disorder such as psoriasis or dermatitis in a patient in need thereof comprising administering to said patient, preferably a human, an effective amount of at least one compound of formula (I) and simultaneously, in parallel, separately or sequentially administering to said patient at least one compound (B), wherein formula (I) and compound (B) are defined in claim 1.

18. A method of treating such as, reducing symptoms of, or preventing a skin disorder such as psoriasis or dermatitis, in a patient in need thereof comprising:
(i) identifying a patient who has received either a compound of formula (I) or a compound (B) respectively; and
(ii) administering to said patient an effective amount of either at least one compound (B) or at least one compound of formula (I) so that said patient is administered with both a compound of formula (I) and a compound (B), wherein formula (I) and compound (B) are defined in claim 1.

19. A method of treating, such as reducing symptoms of, or preventing a skin disorder such as psoriasis or dermatitis in an animal subject in need thereof comprising administering to said animal an effective amount of a composition as claimed in claim 1.

20. A method of treating, such as reducing symptoms of, or preventing a skin disorder such as psoriasis or dermatitis in an animal subject in need thereof comprising administering to said animal an effective amount of at least one compound of formula (I) and simultaneously, in parallel, separately or sequentially administering to said animal at least one compound (B), wherein formula (I) and compound (B) are defined in claim 1.

21. The method of claim 19, wherein the animal subject is a rodent, monkey, or a pig.

22. The method of claim 20, wherein the pharmaceutical composition or the effective amount of compound of formula (I) and compound (B) is used as a positive control.

23. (canceled)

24. The pharmaceutical composition of claim 1 comprising calcipotriol or tacalcitol or a salt, hydrate or solvate thereof optionally in combination with one or more additional secosteroids.

25. The pharmaceutical composition as claimed in 24, wherein the additional secosteroid is selected from the group consisting of alfacalcidol, calcifediol, calcitriol, calcitroic acid, cholecalciferol, dihydrotachysterol, 24,25-dihydroxycholecalciferol, eldecalcitol, ergocalciferol, falecalcitriol, paricalcitol, previtamin D3, tacalcitol/calcipotriol, 22-dihydroergocalciferol, sitocalciferol or pharmaceutically acceptable salts, or hydrates or solvates thereof.

26. A pharmaceutical composition as claimed in claim 1 in a form suitable for topical administration, e.g. a cream, gel, foam or ointment.

27. A method of treating, such as reducing symptoms of, or preventing inflammation of the skin in an animal subject in need thereof comprising administering to said animal an effective amount of a composition as claimed in claim 1.

28. The method of claim 27, comprising the step of reducing the expression one or more of prostaglandin E2 (PGE2), interleukin-1beta (IL-1β), tumor necrosis factor (TNF) or interleukin-6 (IL-6).

29. The method of claim 27, wherein the animal subject has or is suspected of having psoriasis or dermatitis.
